(19) United States
(12) Patent Application Publication
(10) Pub. No.: US 2007/0053491 A1
(43) Pub. Date: Mar. 8, 2007

(54) Adaptive radiation therapy method with target detection

(75) Inventors: Jay S. Schildkraut, Rochester, NY (US); Shoupu Chen, Rochester, NY (US)

Correspondence Address: Mark G. Bocchetti;Patent Legal Staff, Eastman Kodak Company, 343 State Street, Rochester, NY, 14650-2201, US

(73) Assignee: Eastman Kodak Company

(21) Appl. No.: 11/221,133

(22) Filed: Sep. 7, 2005

(51) Int. Cl. A61N 5/10; A61B 5/05

(52) U.S. Cl. 378/65; 600/427

(57) ABSTRACT

A method for delivering radiation therapy to a patient using a three-dimensional planning image for radiation therapy of the patient wherein the planning image includes a radiation therapy target includes the steps of: determining a digitally reconstructed radiograph from the planning image; identifying a region of the target's projection in the digitally reconstructed radiograph; capturing a radiographic image corresponding to the digitally reconstructed radiograph; identifying a region in the captured radiographic image; comparing the region of the target's projection in the digitally reconstructed radiograph with the identified region in the captured radiographic image; and determining a delivery of the radiation therapy in response to this comparison.

### CROSS REFERENCE TO RELATED APPLICATIONS

Reference is made to commonly-assigned copending U.S. application Ser. No. 11/039,422, filed Jan. 20, 2005, entitled RADIATION THERAPY METHOD WITH TARGET DETECTION, by Schildkraut et al., the disclosure of which is incorporated herein.

### FIELD OF THE INVENTION

The invention relates generally to radiation therapy systems, and in particular, to the detection of the target at the time or radiation treatment without the use of internal markers.

### BACKGROUND OF THE INVENTION

In the application of radiotherapy to extra-cranial tumors, movement of the tumor target can result in a decrease in the radiation dose received by the tumor and an increased dose to normal tissue. This is especially relevant to pulmonary tumors that move due to respiration. The uncertainty in the location of the tumor target can be compensated for in several ways. One approach is to increase the treatment margin around the gross tumor volume. This approach increases the probability that all of the tumor will receive a lethal dose of radiation. Unfortunately, it also increases collateral damage to healthy tissue.

If the location of a tumor target could be determined immediately before or during radiation treatment the radiation dose could be concentrated more effectively at the tumor target and less normal tissue would be irradiated. Radiographic images of the patient, in the vicinity of the target, may be captured during radiotherapy. Unfortunately, real-time detection of a tumor target in a projection radiograph is generally very difficult because the target is obscured by overlapping anatomical structures. A number of inventions have been directed at solving this problem.

U.S. Pat. No. 6,731,970 discloses the use of metal as a marker for target tracking. A metal marker has high contrast relative to human tissue in a radiograph. This allows its location to be easily detected by manual or automatic means. However, this approach has significant drawbacks including the requirement of an invasive procedure to implant a marker and the motion of the marker and tumor may not be perfectly correlated. In U.S. Pat. No. 6,898,456 the degree of lung filling is determined from a radiograph by the measurement of the position of the diaphragm, which is clearly visible, relative to the position of stationary anatomy. The lung filling value, instead of the location of an implanted marker, is correlated with the target position.

A number of inventions are directed at performing three-dimensional (3-D) imaging at the time of radiotherapy. U.S. Pat. No. 6,914,959 discloses a combined computed tomography (CT) and radiotherapy system. CT is used to obtain an image for treatment planning and images of the target during treatment. U.S. Pat. No. 6,198,957 discloses a combined magnetic resonance (MR) imaging and radiotherapy system. A drawback to 3-D imaging approaches to target detection during radiotherapy is that the time required to capture and analyze the image may preclude near real-time detection. Also, the addition of a 3-D medical imaging system to a radiotherapy unit would greatly increase its cost. These drawbacks can be mitigated by an approach that captures less than full 3-D images. In U.S. Pat. No. 6,778,850 a plurality of two-dimensional (2-D) x-ray images are captured and synthesized into a low clarity 3-D image.

U.S. Patent Application Publication Nos. 2005/0054916, 2005/0053267, and 2005/0053196 describe a method in which a time sequence of reference fluoroscopic images is captured of an area containing a radiation therapy target throughout a physiological cycle. Moving content in a reference image is enhanced by comparing it to previous images in the sequence. For example, a different image is created from an image by subtracting from it a weighted average of a number of previous images. At the time of radiation treatment a sequence of fluoroscopic images is captured using the same source and imaging device configuration as for the reference images. Moving content is enhanced as before, and the treatment images are correlated with templates that are created from the enhanced reference images. A high correlation between an image and a reference template determines the patient's current position in the physiological cycle.

U.S. Patent Application Publication No. 2004/0092815 describes a method that, instead of directly attempting to detect the location of the target at treatment time, determines the current respiratory state, shift, and orientation of the target region. This information is then used to infer the location of the target. In the planning phase, 3-D CT images are captured in at least two respiratory states preferably including maximum and minimum inhalation. Addition CT images at intermediate respiratory states are estimated from the captures CT images. The location of the target is identified in each of the CT images. Next, a set of digitally reconstructed radiographs (DRR) is calculated for each of the CT images. During radiotherapy radiographs are captured of the target region. The captured radiographs are matched to the set of DRR. When a match to a DRR is found, the respiratory state at the time of capture is determined to be that of the CT image from which the matching DRR was generated. The source and imaging plane location that was used in the calculation of the matching DRR can be used to determine the position and orientation of the target region relative to the position of the radiographic unit. Finally, since the location of the target in the DRR is known, the location of the target at the time the radiograph was captured can be determined.

An object of this invention is to directly detect the location of a target at the time of radiotherapy without the need for the acquisition of multiple 2-D or 3-D images in the planning phase of radiation therapy. Another object of this invention is to directly detect the location of a target at the time of radiotherapy in a way that is fast, does not add significantly to the radiation dose to normal tissue, and does not require major additions to the radiation therapy system. This invention provides a method to detect the location of a radiotherapy target based on identification of the target's projection in a captured radiographic image.

### SUMMARY OF THE INVENTION

Briefly, according to one aspect of the present invention a method for delivering radiation therapy to a patient using a three-dimensional (3-D) planning image for radiation therapy of the patient wherein the planning image includes a radiation therapy target includes the steps of: determining a digitally reconstructed radiograph from the planning image; identifying a region of the target's projection in the digitally reconstructed radiograph; capturing a radiographic image corresponding to the digitally reconstructed radiograph; identifying a region in the captured radiographic image; comparing the region of the target's projection in the digitally reconstructed radiograph with the identified region in the captured radiographic image; and determining a delivery of the radiation therapy in response to this comparison.

This invention builds on U.S. patent application Ser. No. 11/039,422, which discloses a method of real-time target detection in radiotherapy that solves the problem of detecting a target in a 2-D captured radiographic image in two ways: 1) The capture configuration for a radiograph at treatment time is based on an analysis of digitally reconstructed radiographs (DRR) that are generated from a 3-D planning image. This analysis determines capture conditions for which the target can be directly detected. 2) Powerful image processing techniques are used that enable target detection in the presence of superimposed anatomical structures.

This invention provides a method of identifying the region in a captured radiographic image that corresponds to the region of the target's projection in the image. This is accomplished by first, in the planning phase, determining processing conditions that result in the identification of the region of the target's projection in a DRR. A region is identified in the DRR by a method of image segmentation. The identified region is compared with the target's projection in this image. The segmentation process is optimized until the identified region and the target's projection are substantially the same. In the treatment phase, the optimized segmentation procedure is applied to a captured radiographic image in order to identify a region at or near the isocenter. Characteristics of the region identified in the DRR are compared with those of the region identified in the captured radiographic image. Based on this comparison, the probability that the identified region in the captured radiographic image is the target is determined. This probability and the location of the identified region in the captured radiographic image are used to modify the delivery of therapeutic radiation.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a view of a prior art radiation therapy apparatus with target location detection.

FIG. 2 illustrates a prior art method of radiation therapy with target location detection.

FIG. 3 illustrates the method of target location detection.

FIG. 4 illustrates the method of region segmentation.

FIG. 5 shows images from the planning and treatment phases in the method of target location detection.

FIG. 6 illustrates a tumor target's projection in maximal digitally reconstructed radiographs determine with a range of X-ray source positions.

FIG. 7 illustrates the calculation of gradient-based features in a region.

FIG. 8 is a flowchart illustrating an embodiment of target detection.

FIG. 9 is a graph illustrating two images with target displacement.

FIG. 10 is a flowchart illustrating the target displacement finding method of the present invention.

### DETAILED DESCRIPTION OF THE INVENTION

The following is a detailed description of the preferred embodiments of the invention, reference being made to the drawings in which the same reference numerals identify the same elements of structure in each of the several figures.

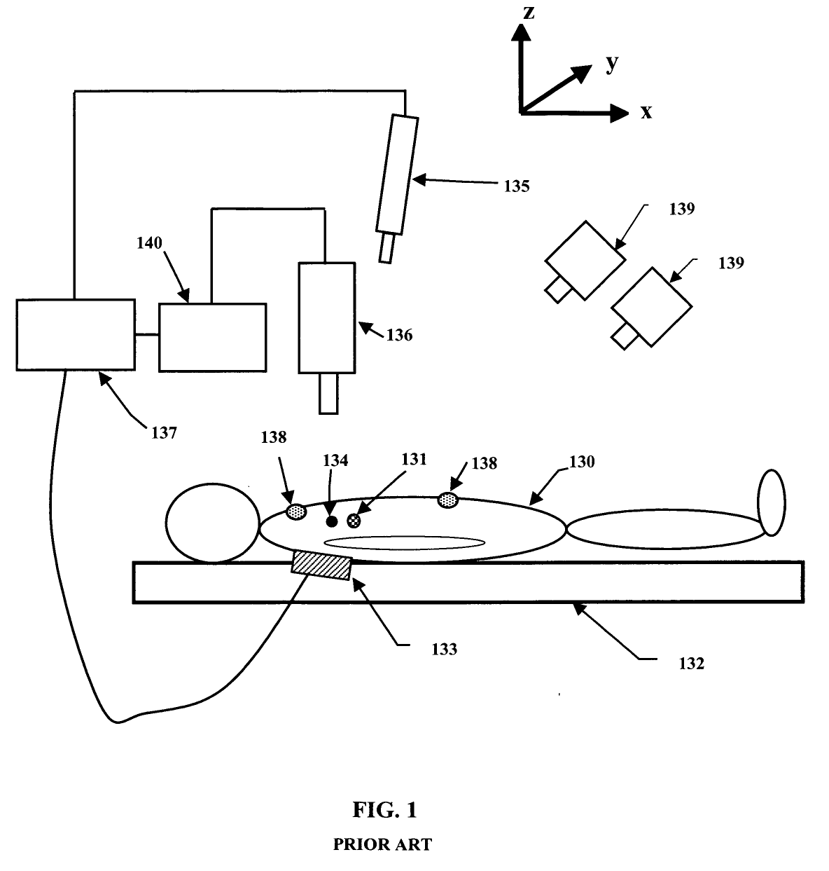

FIG. 1 shows an exemplary radiation therapy system with automatic target location detection. Referring to FIG. 1, a patient 130 is positioned on a support member such as a treatment couch 132. The patient has two or more external markers 138 attached. The position of the external markers is monitored with cameras 139. A therapeutic radiation source 136 is aimed at a isocenter 134 throughout treatment.

A radiography unit is comprised of a diagnostic X-ray source 135 and digital X-ray imaging device 133 images the region of the target 131. The radiation therapy system preferably has more that one radiography unit to enable the location of the target in three-dimensions.

The system has means to accurately determine the position and orientation of the radiography unit relative to the radiotherapy coordinate system. This can be accomplished, for example, with the use of markers placed on the X-ray source and imaging device that are detected by the cameras 139. Another means is to use a phantom that contains markers that are detected by both the cameras and the radiography unit.

The target detection and control unit 137 in FIG. 1 provides a variety of functions. It arranges the radiography units to capture images in which the detection of the target is facilitated. It causes the radiography units to capture images immediately before and during treatment. It determines the location of the target in the captured radiographs relative to the radiotherapy coordinate system in which the isocenter is defined. It further provides information to the radiation therapy control unit 140 that can be used in several ways. The information can be used to decide if radiation therapy should commence or not. The information can be used to decide if radiation therapy should continue or be stopped. It can be used to reposition the patient or the therapeutic radiation source so that the target is at the isocenter.

In an embodiment of this invention, the therapeutic radiation source 136 is used in place of or in addition to the X-ray source 135 to capture radiographic images.

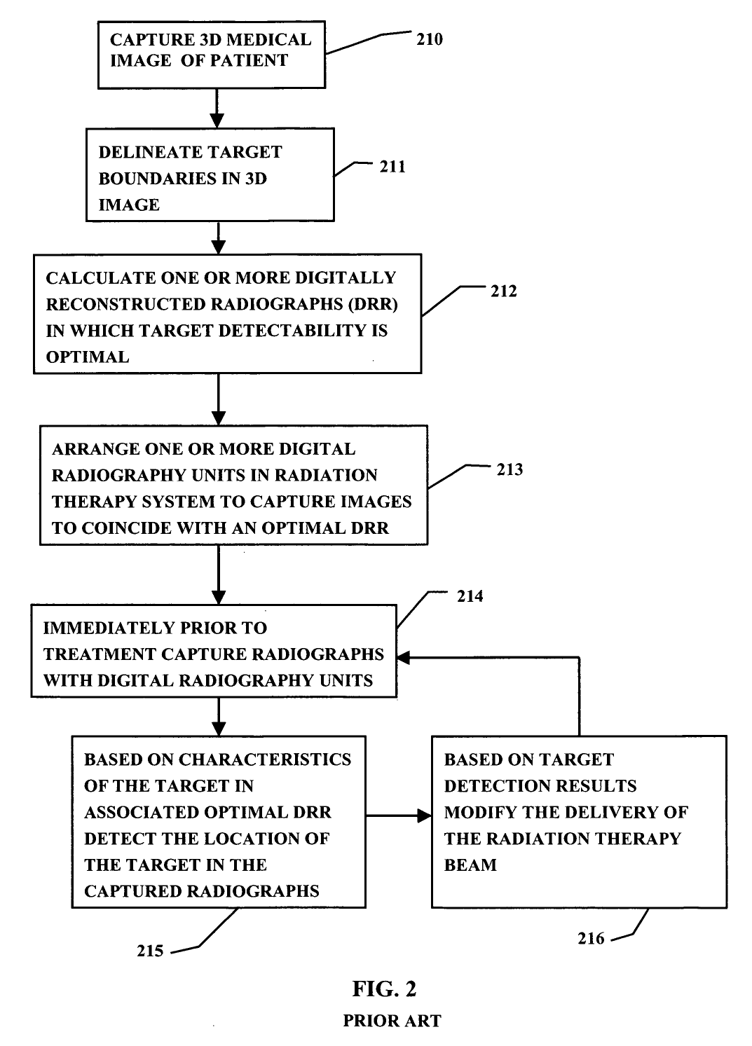

A method of radiation therapy with target detection in accordance with the present invention is diagrammed in FIG. 2. The process begins with step 210 wherein a planning image is captured of the patient. Medical imaging modalities that can be used for this purpose include computed tomography (CT), magnetic resonance imaging (MRI), positron emission tomography (PET), PET-CT, ultrasound, and the like. In step 211, an operator, possibly with the aid of image segmentation software, delineates the boundary of the target volume.

The purpose of step 212 is to determine the best capture conditions for radiographs that are acquired in step 214. In step 212, digitally reconstructed radiographs (DRR) are calculated from the planning image. One or more DRR for which target detection is facilitated is determined. Generally target detection is facilitated when overlap of the target with other anatomy is minimized and the boundary of the target is distinct.

In step 213, one or more radiographic units are arranged to capture images that correspond to a DRR as determined in step 212.

Step 214 occurs immediately before patient exposure with the radiation therapy beam. An image is captured with each of the radiographic units as shown in FIG. 1 by the diagnostic X-ray source 135 and digital X-ray imaging device 133.

In step 215 in FIG. 2, the target is detected in the radiographs captured using the radiographic units. Detection of the target in two or more radiographs enables the localization of the target in three dimensions.

In step 216, the delivery of therapeutic radiation is modified based on the results of step 215. Modification options include, but are not limited to, administering the dose, refraining from administering the dose, repositioning the patient, redirecting the therapeutic radiation beam, and modifying the therapeutic radiation beam. If the modification includes repositioning, redirecting, or modifying, the dose can be administered after the repositioning, redirecting, or modifying.

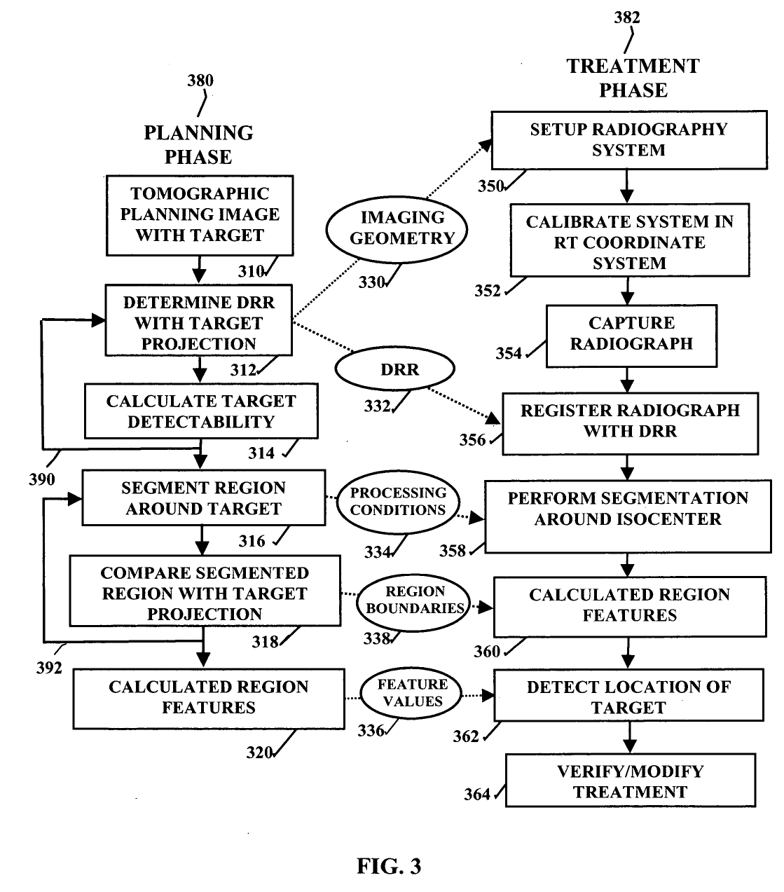

This invention is described in greater detail with reference to FIG. 3. This figure shows the steps in the planning phase 380 and treatment phase 382, the optimization loops 390 and 392 in the planning phase, and the exchange of information between the planning and treatment phase 330, 332, 334, 336, and 338.

A tomographic image of the patient is captured in step 310 that includes the target. The target volume is designated in this image by manually or automatic means. When the target is a tumor this volume is called the gross tumor volume (GTV).

In step 312 a digitally reconstructed radiograph (DRR) is determined from the tomographic image for a chosen imaging geometry which is defined by the location of a virtual X-ray source and image plane relative to the tomographic image. The region of the target's projection in the DRR is mapped by recording which rays, that travel from the source to a pixel in the imaging plane, pass through the target volume. In step 314 the detectability of the target is measured by considering the overlap of the target with other anatomy, the distinctiveness of the target's border, and other detectability metrics.

A metric for the overlap of the target with other anatomy is defined by the equation,

$$Overlap = {\int }_{Target Projection} \frac{{D}_{total} - {D}_{target}}{{D}_{total}} ⅆ A$$

where Dₜₒₜₐₗ is the total integrated density along a ray form the X-ray source to a pixel in the imaging plan and D_{target} is the integrated density only for the target volume. The integral is over the region of the target's projection in the image. The value of this overlap metric ranges between 0.0 for no overlap and 1.0 when the contribution from the target is negligible.

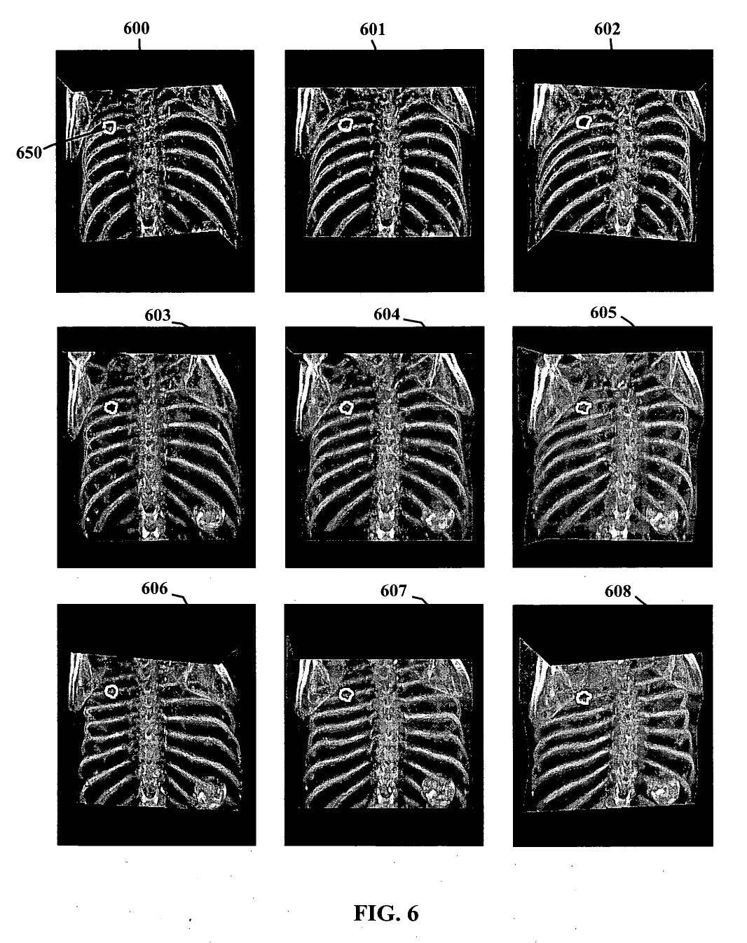

Optimization loop 390, which includes steps 312 and 314, serves to determine preferred imaging geometry 330 for which target detectability is facilitated. FIG. 6 shows images that illustrate this optimization process. DRR were determined from a CT image of a patient with a medium size nodule in the right upper lung. The images 600, 601, 602, 603, 604, 605, 606, 607, and 608 in FIG. 6 show “maximal” DRR that were determined over a range of X-ray source and imaging plane positions. In this calculation the source and imaging plane were arranged on a C-arm. High density anatomy (mostly bone) is emphasized in the DRR by setting a pixel value equal to the maximum X-ray density along a ray from the source to the pixel instead of the integrated density. Furthermore, in each image a white line shows the boundary of the target's projection. For example, line 650 is the outline of the region of the target's projection in image 600. The overlap metric, which is defined above, ranges from 0.964 for image 608 in which the target is partially obscured by a rib to 0.916 for image 603 in which the tumor target's projection is located between ribs.

The purpose of the next two steps 316 and 318 in FIG. 3 are to determine preferred processing conditions 334 that result in automatic segmentation of the target in the DRR. These processing conditions are used later in step 358 in the treatment phase 382 to automatically segment a region in a captured radiographic image. The segmentation of a region in an image generally means to identify a continuous region with a common characteristic. In this invention, the object of segmentation is to identify a region with the common characteristic of being within the target's projection in the image. The success of the segmentation step 316 is measured in step 318 where the segmented region is compared with the region of the target's projection in the DRR. A metric that is used to judge the quality of segmentation of the target's projection is defined by the equation,

$${Q}_{seg} = \frac{1}{2} [ 2 - \frac{{A}_{seg}^{outside}}{{A}_{seg}} - \frac{{A}_{target} - {A}_{seg}^{inside}}{{A}_{target}} ]$$

where A_{target} is the area of the region of the target's projection, A_{seg} is the area of the segmented region, A_{seg}^{outside} is the area of the segmented region that is outside the target's projection, and A_{seg}^{inside} is the area of the segmented region that is inside the target's projection. The value of Q_{seg} ranges between 1.0 when the region of the target's projection and segmented region are identical and 0.0 in the case that the two regions do not overlap.

The purpose of the processing loop 392 is to optimize the segmentation process so that Q_{seg} is as large as possible. The boundary of the region of the target's projection and segmented region 338 are passed to step 360 in the treatment phase.

In step 320 features are calculated for the region of the target's projection and/or the segmented region in the DRR. These features include, but are not limited to, the features that are described in U.S. patent application Ser. No. 11/187,676 which is incorporated into this invention by reference. The value of these features 336 is used subsequently in the treatment phase 382 wherein step 362 they are compared with feature values that are calculated for an identified region in a captured radiographic image in step 360.

The planning phase optimization processes 390 and 392 have the purpose of determining imaging geometry and processing conditions that enable detection of the target at treatment time. Any optimization procedure can be used for this purpose including genetic algorithms, dynamic programming, and gradient decent methods. In one embodiment of this invention the processing loops 390 and 392 are merged in order to jointly optimize imaging geometry and target segmentation processing conditions.

In the treatment phase 382, in step 350 a radiography unit is setup to capture a radiographic image based on the preferred imaging geometry 330 that was determined in the planning phase in steps 312 and 314. This means that the location of the X-ray source and X-ray imaging device of the radiography unit in relation to the patient corresponds with the location of the virtual source and imaging plane relative to the tomographic image in the determination of the DRR in step 312.

Step 352 is a procedure in which the radiography system is calibrated in the coordinate system of the radiotherapy unit. A phantom may be used for this purpose. As a minimum, the location that a line from the X-ray source that passes through the treatment isocenter intersects the X-ray imaging device is determined. This point is referred to as the isocenter's projection in the captured radiographic image.

In step 354 a radiographic image of the target region in the patient is captured by the radiography unit. This may occur immediately before or during irradiation with the treatment beam.

In step 356 the captured radiographic image is registered with the DRR 332 that was determined using the preferred imaging geometry 330. This step is not always necessary. For example, if the patient's conformation at the time of capture of the planning tomographic image and treatment is the same the DRR and captured radiographic image will be similar. Otherwise, registration is necessary so that the region segmentation processing conditions that were optimized in loop 392 base on the appearance of the target in the DRR will successfully segment the target's projection in the captured radiographic image.

In step 358 the preferred processing conditions 334 from step 316 are used to identify a region in the captured radiographic image by region segmentation. In a preferred embodiment of this invention, this region contains the projection of the isocenter in the image.

In step 360 features of the identified region in the captured radiographic image are calculated. The boundaries of the region of the target's projection and segmented region in the DRR 338 are provided to this step because they are used in the calculation of certain features.

In step 362 the value of the features for the identified region in the captured radiographic image are compared with the values 336 that were calculated in step 320 in the planning phase. In one embodiment of this invention, the feature values 336 are for the region of the target's projection in the DRR. In another embodiment of this invention, the feature values 336 are for the segmented region in the DRR that was determined in step 316. The probability that the segmented region in the captured radiographic image is the target and its precise location in the image is determined in this step. Any method of statistical pattern recognition can be used in this step including a neural network, learning vector quantizer (LVQ), support vector machine, and methods that are considered by Anil et al. in “Statistical Pattern Recognition: A Review,” IEEE Transactions on Pattern Analysis and Machine Intelligence, Vol. 22, No. 1, 2000, pp. 4-37.

Finally, in step 364 the location of the identified region in the capture radiographic image and the probability that it is the projection of the target are potentially used in a variety of ways. This information can be used to verify that the target is located within a specified area during treatment. If target location verification fails the treatment beam is stopped. The information can be used for gated radiotherapy. Treatment is commenced only if the target is detected within specified area. The information can be used to relocate the treatment beam and/or the patient so that the target treatment beam is correctly aimed at the target.

In a preferred embodiment of this invention, the target is located in three dimensions by using the method that is described in FIG. 3 to detect the location of the target co-currently in more than one captured radiographic image. The planning phase procedure 380 is performed wherein step 312 different allowed range of X-ray source and/or imaging device location is used in order to generate multiple preferred views of the target. For each planning phase procedure 380 a corresponding treatment phase procedure 382 is used to detect the target in a captured radiographic image.

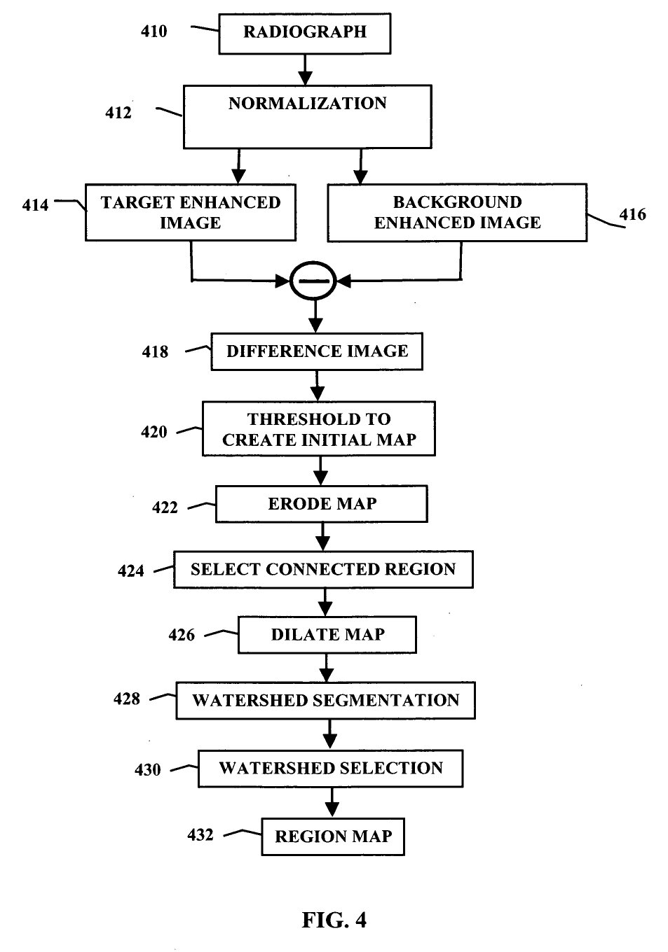

FIG. 4 shows a preferred method of segmenting a region. This method is used in step 316 in FIG. 3 with a DRR as input and in step 358 with a captured radiographic image as input. Referring to FIG. 4, an image 410, whole or in part, is input to the method. In step 412 the input image is normalized to minimize the variation between input images. For example, the image is scaled to obtain a nominal code value mean and standard deviation. Low frequency density gradients may also be removed. In step 414 the image is filtered in order to enhance the target and/or decease background content. Conversely, in step 416 the image is filtered to enhance background content and/or decrease the target. In step 418 the background enhanced image is subtracted from the target enhanced image. The result is a difference image in which the target region has code values that are greater then zero whereas the code values outside of the target region are predominantly less than zero. In step 420 a threshold is applied to the image to create an initial region map. The threshold is normally a small positive number relative to the code value range of the image after step 412.

The region map that was created in step 420 is usually not an accurate map of the target's projection because the image generally contains background features with characteristics of the target. Also, since the input image is a DRR or radiograph other content is superimposed on the target. These problems are corrected by the next series of steps. In step 422 the region map is eroded in order to breakup connections between the target region and other regions. Binary morphological operations as described by J. Serra, in “Image Analysis and Mathematical Morphology,” Vol. 1, Academic Press, 1982, pp. 34-62 are used for this purpose.

In step 424 the connected region in the map that contains the point-of-interest (POI) is retained and all other regions are removed. When the input image is the DRR the POI is the center of the target's projection. In the case that the input image is a capture radiographic image, the POI is the isocenter's projection. Next, in step 426 the selected region is dilated in order to reverse the affect of step 422 on the map of the selected region.

The region map at this point usually still contains the target plus overlapping anatomical structures and therefore needs to be further refined. The region map after step 426 serves as a region of support for the steps that follow. In step 428 a watershed segmentation algorithm is applied to the image in the region of support. Watershed segmentation is described by Vincent and Soille in “Watersheds in Digital Spaces: An Efficient Algorithm Based on Immersion Simulations,” IEEE Trans. Patt. Anal. Machine Intell., Vol. 13, No. 6, 1991, pp. 583-598. In the next step 430 the watershed that contains the POI is retained along with other watersheds that satisfy a connectivity condition. The map of the selected watersheds forms the final region map 432.

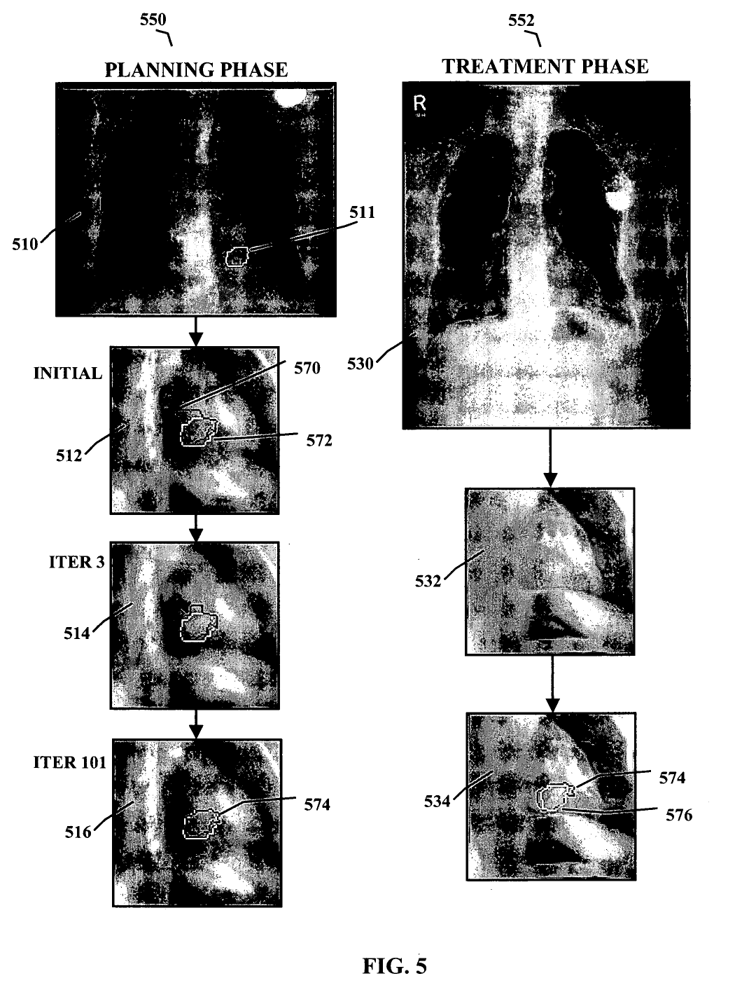

FIG. 5 further describes an embodiment of this invention. On the left is a sequence of images 550 that illustrate the planning phase. Image 510 is a DRR that was determined from a CT of a patient with a pulmonary nodule. A white outline 511 indicates a tumor target's projection in this image.

Referring to FIG. 3, the first time the segmentation step 316 is applied initial conditions for the region segmentation method in FIG. 4 need to be set. This is done by an analysis of the target's projection 511 in FIG. 5. In a embodiment of this invention, gray scale morphological opening operations are used in steps 414 and 416 in FIG. 4 in order to produce a target enhanced and background enhanced image, respectively. Grayscale morphologic operation are described by J. Serra, in “Image Analysis and Mathematical Morphology,” Vol. 1, Academic Press, 1982, pp. 424-478. A Gaussian grayscale morphological template is selected for use in step 414 that has a width close to the width of the target's projection. Long narrow grayscale morphological templates with a range of orientation are selected for use in step 416 of the segmentation method. The templates in step 416 are designed to have a length that exceeds the width of the target's projection so that the target is absent in the filtered image. As a result the difference image that is produced in step 418 will contain positive code values in the target region. In a similar way, all of the steps of the segmentation method in FIG. 4 are initially set to produce a segmented region that closely corresponds to the target's projection.

The result of the first application of step 316 on the DRR is shown in image 512 of FIG. 5. In image 512 the boundary of the segmented region is indicated by a black line 570 and the boundary of the target's projection by the white line 572. Images 514 and 516 in FIG. 5 show the boundaries after 3 and 101 optimization iterations, respectively. This optimization loop is indication by line 392 in FIG. 3. Comparison of the segmentation boundary in images 512, 514, and 516 shows that the correspondence between the segmented region and the target's projection is improved by the optimization process. The black boundary 574 in image 516 shows the best target region segmentation that is obtained by the optimization procedure.

On the right side of FIG. 5 is a sequence of images 552 that illustrate the method of this invention in the treatment phase. Image 530 is a captured radiographic image. Image 532 is a sub-image of image 530 that is centered at the projection of isocenter in the radiograph and registered with the DRR image 510 in the region of the target's projection. In image 534 the white line 576 shows the result of applying the segmentation method in FIG. 4 using the preferred processing conditions 334 to the registered section of the captured radiographic image 532. The black boundary 574 is the boundary that was previously obtained in the planning phase (see image 516) by optimizing the process of segmenting the target's projection in the DRR.

In step 320, of the planning phase, in FIG. 3 the value of features are calculated for the segmented region 574 in image 516 in FIG. 5. In step 360, of the treatment phase, in FIG. 3 the value of features are calculated for the segmented region 576 in image 534 in FIG. 5. These two feature vectors are compared in step 362 to obtain a probability that the identified region in the captured radiographic image 576 is the target.

Region-based features that are calculated in step 320 in the planning phase and 360 in the treatment phase in FIG. 3 are now described in detail. The features that are described are based on region shape, statistics, gradient, texture, and surface. Other features that are know in the art for the purpose of region classification and identification can also be used. While the shape-based feature only requires the region boundary, most other features also require the code values in the region and sometimes those outside of the region. For this reason, a feature may have several values depending on the version of the image that was used in the calculation. For example, the feature can be calculated for the raw input image which in this invention is either a DRR or a captured radiographic image. Preferentially, features are calculated using the code values of the normalized image from step 412 and/or the difference image from step 418 of the method of region segmentation that is illustrated in FIG. 4.

The feature Shape₁ compares an identified region to a reference region. In step 320 the identified region is the region that is segmented in the DRR in step 316 and the reference region is the target's projection in the DRR. In step 360 the identified region is the region that is segmented in the captured radiographic image in step 358. The reference region is either the target's projection or the segmented region in the corresponding DRR 332 which is provided by the region boundaries 338. The definition of this feature is,

$${Shape}_{1} = \frac{1}{2} [ 2 - \frac{{A}^{outside}}{A} - \frac{{A}_{ref} - {A}^{inside}}{{A}_{ref}} ]$$

where A is the area of the identified region, A_{ref} is the area of the reference region, A^{outside} is the area of the identified region that is outside the reference region, and A^{inside} is the area of the identified that is in side the reference region.

A statistics feature compares the average code value in a region to the average in a surrounding region. This feature is defined by
Stat₁=μ_{region}−μ_{surround}
where μ_{region} and μ_{surround} are the mean code value of the region and surrounding region, respectively. Other statistical measures can also be used including the code value standard deviation, minimum code value, and maximum code value.

Gradient-based features are valuable in the detection of tumor targets in an X-ray image. The gradient direction at pixels that are within the region of the target's projection tend to converge to a common point. A single band digital image is a discrete two-dimensional function. A number of linear filters have be developed for the purpose of estimating the first derivative of this function at each pixel location. For example, a Sobel filter is commonly used. The magnitude Mᵢⱼ and direction θᵢⱼ of the gradient at pixel ij in an image is defined by,

$${M}_{ij} = \sqrt{{( \frac{\partial {F}_{ij}}{\partial x} )}^{2} + {( \frac{\partial {F}_{ij}}{\partial y} )}^{2}}$$

$${θ}_{ij} = {tan}^{- 1} [ \frac{\frac{\partial {F}_{ij}}{\partial y}}{\frac{\partial {F}_{ij}}{\partial x}} ]$$

where Fᵢⱼ is the code value.

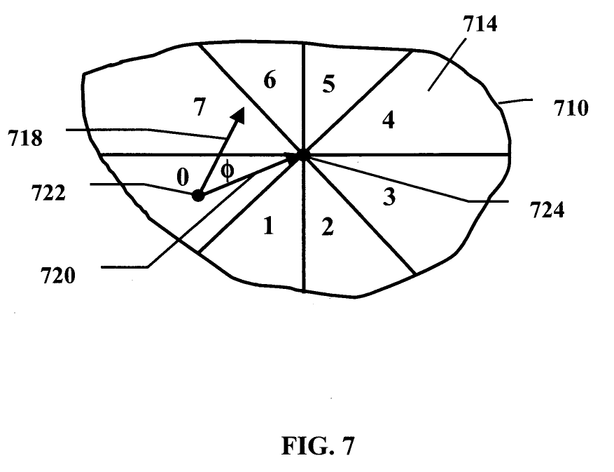

The calculation of a gradient-based feature for a region is described with reference to FIG. 7. The line 710 marks the boundary of the region. The point of origin for the calculation 724 is typically chosen as the geometric center or the location of maximum density in the region. The region is divided into S sections about the origin. In FIG. 7 eight sections are shown which each extend 45 degrees about the origin. For example, 714 in FIG. 7 is section 4 of the region. Consider a pixel 722 in region 710. A line 720 is drawn between the pixel and the origin 724. The line 718 shows the direction of the image gradient at this pixel. The angle between the gradient direction 718 and line 720 is denoted by φᵢⱼ. A measure of the degree that the gradient of pixels in section k point to the origin is expressed by the equation,

$${ψ}_{k} = \frac{1}{{N}_{k}} {\sum }_{section k} cos {ϕ}_{ij}$$

$${t}_{1} \leq {M}_{ij} \leq {t}_{2}$$

where Nₖ is the number of pixels in section k. Only pixels with a gradient magnitude above t₁ and below t₂ are included in the summation. The gradient feature for the region is defined by,

$${Grad}_{1} = \frac{\overline{ψ}}{σ}$$

$$where , \overline{ψ} = \frac{1}{S} {\sum }_{k = 0}^{S - 1} {ψ}_{k}$$

$$σ = \sqrt{\frac{1}{S} {\sum }_{k = 0}^{S - 1} {( {ψ}_{k} - \overline{ψ} )}^{2}}$$

Texture-based features for the region are calculated using the code values, gradient magnitude, or gradient direction in an image. Texture features that are based on the cooccurrence function are describe by Bevk and Kononenko in “A Statistical Approach to Texture Description of Medical Images: A Preliminary Study,” 15th IEEE Symposium on Computer-Based Medical Systems, Jun. 4-7, 2002. Two texture-based features are given by the equations,

$${Texture}_{1} = {\sum }_{ij} {[ C ( i , j ) ]}^{2}$$

$${Texture}_{2} = {\sum }_{ij} {( i - j )}^{2} C ( i , j )$$

where C(ij) is the cooccurrence function calculated over neighboring pixels. The summations range from minimum to maximum code value. The feature Texture₁ is referred to as the energy and Texture₂ as the contrast. Any of the other texture features described by Haralick et al. in “Texture Features for Image Classification,” IEEE Transactions on Systems, Man, Cybernetics, 1973, pp. 610-621, can also be used.

A grayscale image can be interpreted as a relief map in which the code value at a pixel is a measure of the elevation of a surface at that location. Surface-based features are obtained by fitting the image surface in a region to a 4th order bivariate polynomial. The principle curvatures are calculated at the point of highest elevation in the region as described by Abmayr et al. in “Local Polynomial Reconstruction of Intensity Data as Basis of Detecting Homologous Points and Contours with Subpixel Accuracy Applied on IMAGER 5003,” Proceedings of the ISPRS working group V/1, Panoramic Photogrammetry Workshop, Vol. XXXIV, Part 5/W16, Dresden, 2004. Second-order derivatives of the fitted polynomial are calculated to obtain the elements of the Hessian matrix. The maximum and minimum eigenvalue of the Hessian matrix λₘₐₓ and λₘᵢₙ are the principle curvatures. The surface-based region features are,
Surface₁=λₘᵢₙ
Surface₂=λₘₐₓ
Surface₃=λₘᵢₙλₘₐₓ

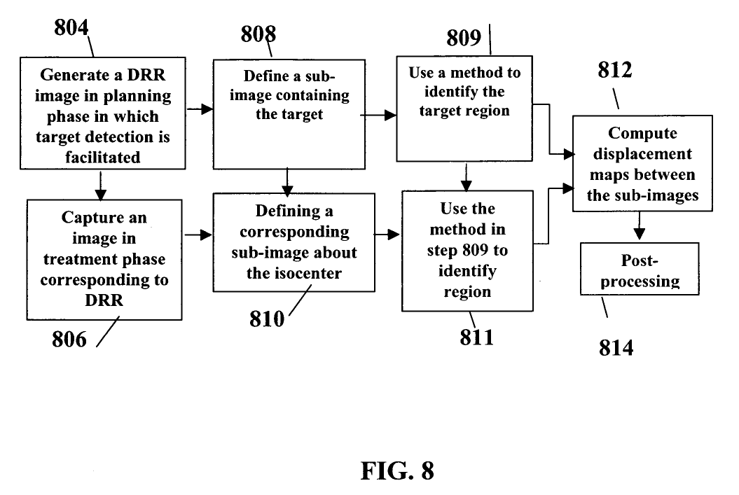

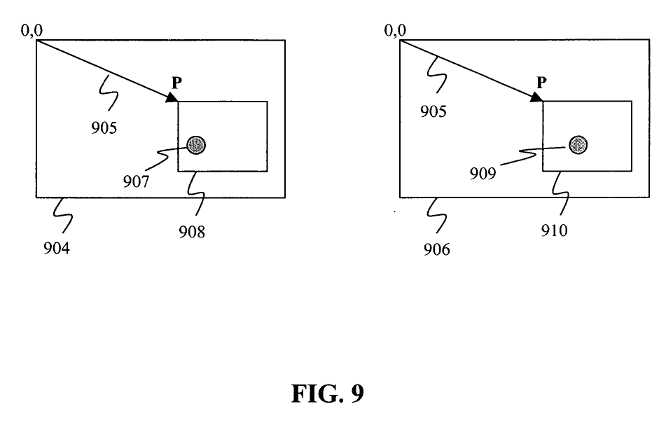

Referring to FIG. 8, another embodiment of target localization of the present invention is illustrated. A planning phase image 804 is a DRR as determined in step 212 in FIG. 2. This image, which is denoted by Iₚ, is illustrated in FIG. 9 as image 904. The projection of the target in this image is illustrated by 907 is shown in image 904.

806 is the captured radiographic image (see step 214 in FIG. 2) from the treatment phase. This image, which is denoted by I_{T}, is shown in FIG. 9 as image 906. An illustrative target position 909 is shown in image 906. Note that target 907 and target 909 are the images of the same tumor in a patient's body but taken at different times.

In practice, images I_{P} and I_{T} have the same size because of the correspondence between the captured radiographic image and the DRR. The origin of these images can be arbitrarily defined at the upper left corner as shown in FIG. 9.

One purpose of target detection of the this invention is to find the target position difference, due to various causes (e.g. respiration), between the planning phase and the treatment phase. Because of the elastic nature of the soft tissue, position difference of a target varies depending on the location of the target within the body. In case of multiple targets, the motion of all the targets is considered non-rigid. While for an individual target, within a smaller region, the motion can be regarded as either rigid or non-rigid. Therefore, a step of defining a target-centric sub-image is employed.

Referring to FIG. 8, step 808 defines a first sub-image (denoted by Ĩ_{P}) that contains a target 907 in image I_{P} 904. The position of sub-image Ĩ_{P} 908 is determined by a vector P 905. In step 810, with the same position vector P, a second sub-image (denoted by Ĩ_{T}) 910 can be defined in image 906. Images 908 and 910 have the same size that can be predetermined by the maximum possible target displacement in an image so that target 909 is enclosed in sub-image 910.

Before applying the method of finding the displacement, target 909 is identified in step 811 where the identification method is identical to that in step 809 in which target 907 is identified in the planning phase.

A method of finding the displacement (step 812) of target 909 from 907 based on gradients is now discussed. The process can be formulated as determining, mathematically, a mapping between the coordinates of a part of an image (e.g. 908) and a part of another image (e.g. 910), such that points in the two sub-images that correspond to the same region are mapped to each other.

For the purpose of discussion, express the sub-image Ĩ_{T} 910 as I(xₜ, yₜ, t) and the sub-image Ĩ_{P} 908 as I(xₜ₊₁, yₜ₊₁, t+1). The notations x and y are the horizontal and vertical coordinates of the image planar coordinate system, and t is the image index. It is important to note that the origin, (x=0, y=0), of the image planar coordinate system is defined at the center of the image plane. The coordinates x and y can be non-integers.

The image (or image pixel) is also indexed as I(i, j) where the indices (i and j) are strictly integers and parameter t is ignored for simplicity. This representation is consistent with indexing a matrix in the discrete domain. If the height of the image is h and the width is w, the corresponding image plane coordinates, x and y, at location (i, j) can be computed as x=i−(w−1)/2.0, and y=(h−1)/2.0−j. The column index i runs from 0 to w−1. The row index j runs from 0 to h−1.

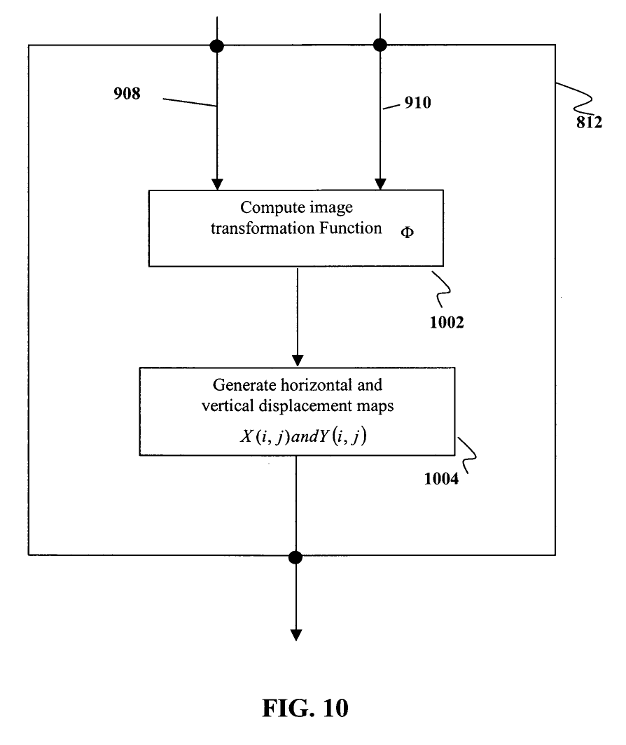

In general, the mapping process is to find an optimal affine transformation function Φₜ₊₁(xₜ, yₜ) (step 1002 in FIG. 10) such that
[xₜ₊₁, yₜ₊₁,1]^{T}=Φₜ₊₁ (xₜ, yₜ)[xₜ, yₜ,1]^{T}
Now, define the energy function (or L₂ norm measure) of the difference between the two images as

$$E ( {Φ}_{t + 1} ) = {\sum }_{x , y ∈ Ω} {( ( I ( {x}_{t} , {y}_{t} , t ) ) - I ( {x}_{t + 1} , {y}_{t + 1} , t + 1 ) )}^{2}$$

where Ω is a spatial neighborhood for a local mapping (non-rigid mapping). For a global mapping, Ω is the entire image.

The transformation function Φₜ₊₁(xₜ, yₜ) is a 3×3 matrix with elements shown as,

$$Φ = [ \begin{matrix}{ϕ}_{00} & {ϕ}_{01} & {ϕ}_{02} \\ {ϕ}_{10} & {ϕ}_{11} & {ϕ}_{12} \\ 0 & 0 & 1\end{matrix} ]$$

This transformation matrix consists of two parts, a rotation sub-matrix

$$[ \begin{matrix}{ϕ}_{00} & {ϕ}_{01} \\ {ϕ}_{10} & {ϕ}_{11}\end{matrix} ]$$

and a translation vector

$$[ \begin{matrix}{ϕ}_{02} \\ {ϕ}_{12}\end{matrix} ] .$$

There are a variety of ways to minimize the energy function. For instance, using derivatives of image pixel in terms of spatial positions and intensity (see “Robot Vision”, B. K. P. Horn, The MIT Press McGraw-Hill Book Company, 1986), optimal values of the entries of the transformation function Φ can be found. The optimized transformation function by applying optimal affine transformation function maps one image to another.

In practice, using the optimized transformation function Φ two displacement maps X(i, j), and Y(i, j) can be generated (step 1004 in FIG. 10). These two maps carry out the mapping process through image interpolation. For both displacement maps, X(i, j) and Y(i, j), the column index i runs from 0 to w−1 and the row index j runs from 0 to h−1.

The steps depicted in FIG. 10 are applicable in step 356 in FIG. 3. Noted also that step 814 in FIG. 8 is equivalent to step 216 in FIG. 2 where treatment verification/modification take place.

The invention has been described in detail with particular reference to a presently preferred embodiment, but it will be understood that variations and modifications can be effected within the scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### Parts List

130 patient
131 target
132 treatment couch
133 digital X-ray imaging device
134 isocenter
135 diagnostic X-ray source
136 therapeutic radiation source
137 control unit
138 external marker
139 camera
140 radiation therapy control unit
210 capture image of patient
211 delineate target boundaries
212 determine best capture conditions
213 one or more radiographic units arranged
214 image captured
215 target detected
216 delivery of therapeutic radiation modified
310 tomographic image of patient captured
312 digitally reconstructed radiograph (DRR) determined
314 detectability of target measured
316 segment region around target
318 compare segmented region with target projection
320 calculate region features
330 imaging geometry
332 digitally reconstructed radiograph (DRR)
334 processing conditions
336 feature values
338 segmented region
350 setup radiograph system
352 radiography system calibrated
354 capture radiograph
356 register radiograph with DRR
358 perform segmentation around isocenter
360 calculated region features
362 detect location of target
364 verify/modify treatment
380 planning phase
382 treatment phase
390 optimization loop
392 optimization loop
410 image
412 image normalized
414 image filtered to enhance target
416 image filtered to enhance background
418 background enhanced image subtracted from target enhanced image
420 threshold to create initial map
422 region map eroded
424 select connected region
426 dilate map
428 watershed segmentation
430 watershed selection
432 final region map formed
510 DRR image
511 target's projection
512 image
514 image
516 image
530 captured radiographic image
532 sub-image of captured radiographic image
534 image
550 planning phase
552 treatment phase
570 segmented region
572 boundary of target projection
574 best target region segmentation in DRR
576 segmented region in captured radiographic image
600 image
601 image
602 image
603 image
604 image
605 image
606 image
607 image
608 image
650 boundary of target projection
710 region
714 section
718 gradient direction
720 line from pixel to point of origin
722 pixel
724 point of origin for calculation
804 planning phase image
806 captured radiographic image
808 define sub-image
809 target is identified
810 defining sub-image around isocenter
811 target is identified
812 computing displacement
814 post-processing
904 image
905 vector P
906 image
907 target position
908 first sub-image
909 target position
910 second sub-image
1002 computing image transformation function
1004 generating horizontal and vertical displacement maps

1. A method for delivering radiation therapy to a patient using a three-dimensional planning image for radiation therapy of the patient wherein the planning image includes a radiation therapy target, the method comprising the steps of:
determining a digitally reconstructed radiograph from the planning image;
identifying a region of the target's projection in the digitally reconstructed radiograph;
capturing a radiographic image corresponding to the digitally reconstructed radiograph;
identifying a region in the captured radiographic image;
comparing the region of the target's projection in the digitally reconstructed radiograph with the identified region in the captured radiographic image; and
determining a delivery of the radiation therapy in response to this comparison.

2. The method of claim 1 wherein the method of identifying a region in the captured radiographic image identifies a region that is substantially the same as region of the target's projection in the digitally reconstructed radiograph.

3. The method of claim 2 wherein the method of identifying a region includes a grayscale morphological operation.

4. The method of claim 2 wherein the method of identifying a region includes applying a threshold.

5. The method of claim 2 wherein the method of identifying a region includes combining connected regions into a single region.

6. The method of claim 2 wherein the method of identifying a region includes splitting a region into multiple sub-regions.

7. The method of claim 2 wherein the method of identifying a region includes a binary morphological operation.

8. The method of claim 2 wherein the method of identifying a region includes watershed segmentation.

9. The method of claim 2 wherein the captured radiographic image is registered with the digitally reconstructed radiograph.

10. The method of claim 1 wherein comparing the region of the target's projection in the digitally reconstructed radiograph with the identified region in the captured radiographic image is based on region location.

11. The method of claim 1 wherein comparing the region of the target's projection in the digitally reconstructed radiograph with the identified region in the captured radiographic image is based on region shape.

12. The method of claim 1 wherein comparing the region of the target's projection in the digitally reconstructed radiograph with the identified region in the captured radiographic image is based on region code value statistics.

13. The method of claim 1 wherein comparing the region of the target's projection in the digitally reconstructed radiograph with the identified region in the captured radiographic image is based on the gradient in the region.

14. The method of claim 1 wherein comparing the region of the target's projection in the digitally reconstructed radiograph with the identified region in the captured radiographic image is based on a surface fit in the region.

15. The method of claim 1 wherein comparing the region of the target's projection in the digitally reconstructed radiograph with the identified region in the captured radiographic image is based on the texture of the region.

16. A method for delivering radiation therapy to a patient using a three-dimensional planning image for radiation therapy of the patient wherein the planning image includes a radiation therapy target, the method comprising the steps of:
(a) determining a digitally reconstructed radiograph image in the planning phase from the planning image;
(b) defining a first sub-image in the digitally reconstructed radiograph;
(c) capturing a radiographic image, in the treatment phase, corresponding to the digitally reconstructed radiograph;
(d) defining a second sub-image in the captured radiographic image;
(e) segmenting a first region in the first sub-image;
(f) segmenting a second region in the second sub-image; and
(g) determining displacement maps between the first region and second region.

17. The method of claim 16 wherein defining a first sub-image in the digitally reconstructed radiograph comprises the steps of:
(a) determining the location of the first sub-image; and
(b) determining the size of the first sub-image such that it contains the projection of the target.

18. The method of claim 16 wherein defining a second sub-image in the captured radiographic image comprises the steps of:
(a) determining the location of the second sub-image corresponding to the location of the first sub-image; and
(b) determining the size of the second sub-image corresponding to the size of the first sub-image such that it contains the projection of the target.

19. The method of claim 16 wherein determining displacement maps between the first region and second region further comprises the steps of:
(a) computing a transformation function between the first sub-image and second sub-image;
(b) calculating displacement maps using the computed transformation function; and
(c) mapping the first sub-image and the second sub-image through image interpolation.

20. A method for delivering radiation therapy to a patient using a three-dimensional planning image for radiation therapy of the patient wherein the planning image includes a radiation therapy target, the method comprising the steps of:
determining a digitally reconstructed radiograph from the planning image;
identifying a region of the target's projection in the digitally reconstructed radiograph;
capturing a radiographic image corresponding to the digitally reconstructed radiograph;
identifying a region in the captured radiographic image; and
comparing the region of the target's projection in the digitally reconstructed radiograph with the identified region in the captured radiographic image.
